(19) United States
(12) Patent Application Publication
(10) Pub. No.: US 2011/0065744 A1
(43) Pub. Date: Mar. 17, 2011

(54) Method Of Inhibiting Mucin Secretion

(75) Inventors: Helmut Albrecht, Parsippany, NJ (US); K. Chul Kim, Albuquerque, NM (US); Bruce Rubin, Winston-Salem, NC (US); Jeanclare Seagrave, Albuquerque, NM (US); Gail Solomon, Parsippany, NJ (US)

(73) Assignee: Reckitt Benckiser Inc., Parsippany, NJ (US)

(21) Appl. No.: 12/558,517

(22) Filed: Sep. 12, 2009

(51) Int. Cl. A61K 31/439; A61K 31/09; A61K 31/137

(52) U.S. Cl. 514/289; 514/719; 514/653; 514/646

(57) ABSTRACT

A method of inhibiting mucus secretion in an individual that includes administering an effective amount of a composition which comprises guaifenesin.

### BACKGROUND OF THE INVENTION

1. Field of the Invention

The present invention relates to the use of a pharmaceutical compound for the inhibition of mucus secretion in an individual. In particular, the present invention relates to the use of guaifenesin for the inhibition of mucus secretion.

2. Description of Related Art

Guaifenesin, whose chemical name is 3-(2-methoxyphenoxy)-1,2-propanediol, is an expectorant. An expectorant is a drug that helps bring up mucus and other material from the lungs, bronchi, and trachea. Guaifenesin is thought to act by thinning the mucus, loosening phlegm and bronchial secretions, and also by lubricating the irritated respiratory tract. By thinning the mucus, guaifenesin reduces the viscosity of the mucal secretions, and as a result increases the efficiency of the cough reflex and of ciliary action in removing accumulated secretions from trachea and bronchi. The effect felt by an individual is that a nonproductive cough becomes more productive and less frequent.

In the prior art there are disclosed methods of inhibiting mucin. However, these methods are directed to the treatment of chronic conditions, such as asthma. WO 2004/043392 discloses a method of modulating mucin synthesis and the therapeutic application of compounds in controlling mucin over-production associated with diseases such as chronic obstructive pulmonary diseases (COPD), including chronic bronchitis, and, inflammatory lung diseases, asthma, cystic fibrosis and acute or chronic respiratory infectious diseases using compounds of a defined formula having at least two aromatic rings.

### BRIEF SUMMARY OF THE INVENTION

The applicant has developed a method of inhibiting the secretion of mucus in an individual which comprises administering an effective amount of a composition which comprises guaifenesin.

According to a first aspect of the present invention there is provided a method of inhibiting mucus secretion in an individual which comprises administering an effective amount of a composition which comprises guaifenesin. The composition can contain from approximately 600 mg-1200 mg of guaifenesin.

The guaifenesin can be administered in many suitable forms such as a tablet, powder, capsule, liquid or liquigel. The guaifenesin can be administered orally.

The mucin can be produced in the upper respiratory tract of an individual.

The composition can contain one or more additional active agents selected from the group including, but not limited to, an antitussive such as dextromethorphan hydrobromide, a decongestant such as phenylephrine hydrochloride, pseudoephedrine hydrochloride or ephedrine, an antihistamine such as chlorpheniramine maleate, brompheniramine maleate, phenindamine tartrate, pyrilamine maleate, doxylamine succinate, phenyltoloxamine citrate, diphenhydramine hydrochloride, promethazine, and clemastine fumerate, fexofenadine or a combination thereof.

The composition can have an immediate release portion and a sustained release portion, such that the inhibition of mucus secretion is therapeutically achieved for a period of approximately 12 hours.

According to a second aspect of the present invention there is provided a method of treating an individual having a disease or condition characterized by increased mucin secretion with an effective amount of a composition which comprises guaifenesin as described in the first aspect of the present invention.

### BRIEF DESCRIPTION OF THE FIGURES

Example embodiments of the present invention will now be described in more detail with reference to the accompanying figures.

FIG. 1 illustrates the treatment protocol.

FIG. 2 is a graph showing the effect of guaifenesin on MUC5AC mucin secretion: 30 min

FIGS. 3a and 3b are graphs showing the effect of guaifenesin on MUC5AC mucin secretion: 6 hours

FIGS. 4a and 4b are graphs showing the effect of guaifenesin on MUC5AC mucin secretion: 24 hours

FIGS. 5a and 5b are graphs showing the effect of guaifenesin on MUC5AC mucin secretion: 48 hours

FIG. 6 is a graph showing the effect of guaifenesin on mucociliary clearance.

FIGS. 7a and 7b are graphs showing metabolic activity.

FIGS. 8a, 8b and 8c are graphs showing mucus rheology.

FIGS. 9a and 9b are graphs showing the vector sum of viscosity and elasticity against time and dose.

### DETAILED DESCRIPTION OF PREFERRED EMBODIMENTS

### Materials and Methods

Cells:

EpiAirway cultures (normal human bronchial epithelial) cells grown on Millipore Transwells, 1 or 4.2 cm² surface area. The cells were purchased from MatTek, and were cultured at air-liquid interface for two (mucus synthesis and secretion) or three (mucociliary transport and mucus rheology) weeks prior to use.

Guaifenesin (GGE) Treatment:

For mucociliary clearance, a stock guaifenesin solution of 2 mg/mL in culture medium was prepared in the morning of each experiment and kept cold until dilution into warmed medium to the target concentrations of 0.2, 2, 20 or 200 μg/mL. The medium in the basolateral compartment of each culture was replaced with the GGE-containing medium, and the cultures were returned to the 37° C., 5% CO₂ incubator for as the times indicated. The experiments were repeated three times on independent cultures.

The concentrations used in the in vitro experiments range from 0.2 μg/mL to 20 mg/mL and thus bracket the clinical doses used in humans.

Measurement of Mucin Secretion:

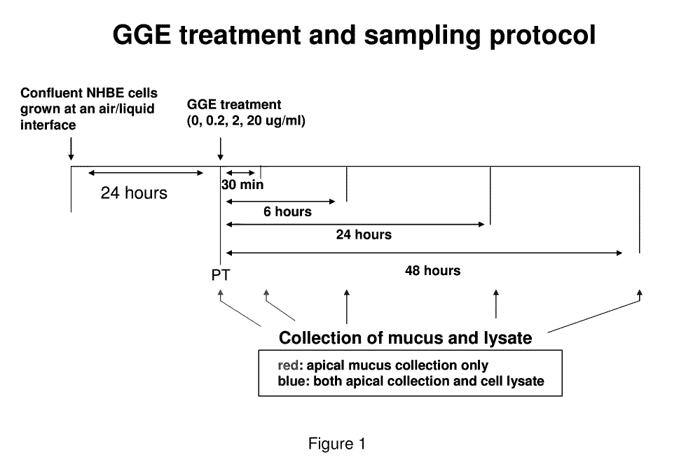

GUAIFENESIN solutions were prepared by dissolving in PBS (phosphate buffered saline) immediately before treatment of the cells. MUC5AC mucins were quantified by ELISA using 45M1 antibody (Labvision, Fremont, Calif.). Confluent 1 cm² NHBE cells grown on an air/liquid interface were washed from the apical surface with 200 μL PBS and incubated with fresh complete growth medium added to the basal chamber. Cultures were incubated 24 hours to collect the apical fluid (pretreatment sample or PT) by adding 100 μL PBS to the apical surface of the cultures. PBS was added to dilute the highly viscous, thin mucus layer on the surface. Because of the small size of the insert, it was not feasible to collect a sufficient amount of mucus for both pharmacology and rheology without the addition of PBS. After collecting 100 μL of the diluted mucus samples (PT), cultures were divided into three groups (6 hr, 24 hr and 48 hr), 16 inserts per group, and treated with varying concentrations of guaifenesin (0, 0.2, 2, 20 μg/mL) for each time group, 4 inserts per each dose. Thus, a total of 48 inserts were used for this study (4 inserts/dose×4 doses/time point×3 time points). The apical fluid was collected at 30 minutes following drug treatment from all the cultures to see whether guaifenesin affects the “secretion” of mucins. The apical mucus sample was collected in two steps—first by adding 100 μL PBS to the apical surface (1^{st} wash) and then by adding 100 μL PBS containing 5 mM dithiothreitol (DTT) (2^{nd} wash). Samples from each wash were assayed for MUC5AC content and the sum of the two values (the 1^{st} and 2^{nd} wash) was expressed as the “released MUC5AC” of the culture. At the three different time points (i.e., 6, 24, and 48 hr), cultures were washed to collect the apical fluid as described above (“released mucin”) and lysed using a lysis buffer (PBS, pH 7.2, 1 mM Triton X-100, 2 mM EDTA, 1 mM PSMF and 5 mM DTT) (“cellular mucin”). The amount of mucin in each sample (either secreted, released or cell lysate) was divided by the amount of mucin in the PT sample collected from the same well in order to obtain a “secretory index” to compensate for the variations among the cultures. The treatment protocol is depicted in FIG. 1.

Measurement of Mucociliary Clearance:

Cultures (4.2 cm²) were exposed to basolateral guaifenesin for 1 or 6 hr. The cultures were removed from the incubator and placed on the stage of digital imaging microscopy system. Video data were collected for 10 seconds using a 25× objective. The rate of movement of endogenous cell debris was analyzed on the video images using a transparent template overlay on the video images and a stopwatch to measure at least 5 particles on each culture, for a total of between 30 and 45 measurements per condition.

Collection of Mucus:

Following the analysis of clearance, mucus was harvested from the apical surface of the cultures, without dilution.

Viability:

The apical surfaces of the cultures were then washed with PBS and the metabolic activity, an indicator of viability, was measured using the Water Soluble Tetrazolium (WST) assay (Boehringer).

Rheologic Measurements:

The rheological properties of apical mucus secretions (20 μL) were measured using an AR1000 controlled stress rheometer (TA Instruments, New Castle, Del.) using a parallel plate geometry. The dynamic linear viscoelastic behavior was determined from the strain response to an oscillating stress and reported as a storage or elastic modulus (G′), and loss or viscous (G″) modulus, as a function of frequency w such that viscosity, η′=G″/ω. Rheologic data can also be presented using vectorial notation as tangent δ which is the ratio of viscosity to elasticity and G*, the vector sum of viscosity and elasticity (mechanical impedance). When stress in the linear range is used to evaluate the materials, the material properties are independent of stress.

In order to conduct a frequency sweep from 0.1 to 1000 rad/s, we evaluated viscoelasticity using a creep test at 0.5 Pa for 2 minutes. The strain response was fitted to a discrete relaxation spectrum, transformed to the retardation spectrum, and then to the storage and loss moduli, as a function of frequency, using methods developed by the PI. We evaluated the linear viscoelasticity at 1 and 100 rad/s and we used an oscillatory stress sweep and steady shear flow experiments to evaluate the behavior in the non-linear ranges. The oscillatory sweep data were analyzed by observing the stress where G′ and G″ crossed. This point indicates where the material shows more viscous behavior (irreversible deformation and flow) than recoil behavior.

All rheologic measurements were made by technicians who were blinded to the treatment group origin.

Statistics:

For mucin secretion, differences between control and guaifenesin treatment groups were assessed by comparing the means using Student's t-test for unpaired samples and p<0.05 was considered significant. All the values in the figures represent means±SEM of 4 cultures unless otherwise stated. * p<0.05, ** p<0.01

For mucociliary clearance, differences between control and guaifenesin treatment groups were assessed by comparing the means using ANOVA, with a Bonferroni post-test to assess differences from the controls tested at the same time after treatment. A p value of <0.05 was considered statistically significant.

For rheology experiments, data were analyzed using the StatView™ 5 statistics package. Raw data were visually confirmed to be normally distributed about the mean. ANOVA was used to compare results of treating sputum with different concentrations of guaifenesin. Fisher's protected least significant difference test was done to determine significance with multiple comparisons. Data are presented as group means±1 standard error unless otherwise indicated. By convention p<0.05 is considered statistically significant.

### Results

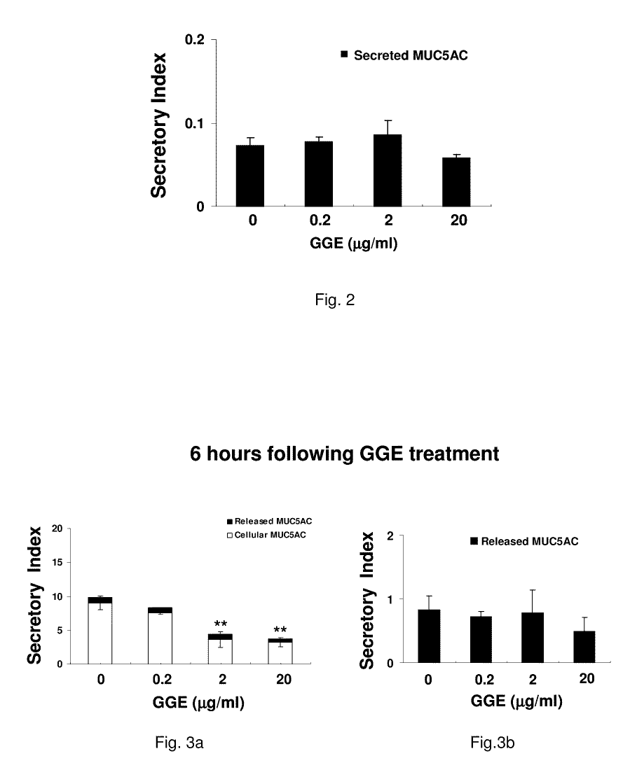

In FIG. 2, EpiAirway cultures were treated with the indicated concentrations of guaifenesin for 30 min. Secreted MUC5AC was compared with the pre-treatment values.

During the 30 minute treatment period, there was no significant difference (p<0.05) between control and guaifenesin treatment groups.

In FIG. 3a, the white boxes represent the amount of mucin associated with the cell, whereas the black boxes represent the amount of mucin released during the given period of treatment. Therefore, the addition of the white box and the black box represents the total amount of mucin produced during the given period. The total amounts of MUC5AC were compared for statistical differences between control (no guaifenesin) and guaifenesin groups.

Treatment of NHBE cells with guaifenesin for 6 hours did not affect the amounts of mucins released (FIG. 3b). However, the total amounts of mucins produced during the 6 hour treatment period were significantly (p<0.01) suppressed by the presence of guaifenesin (both 2 μg/ml and 20 μg/ml).

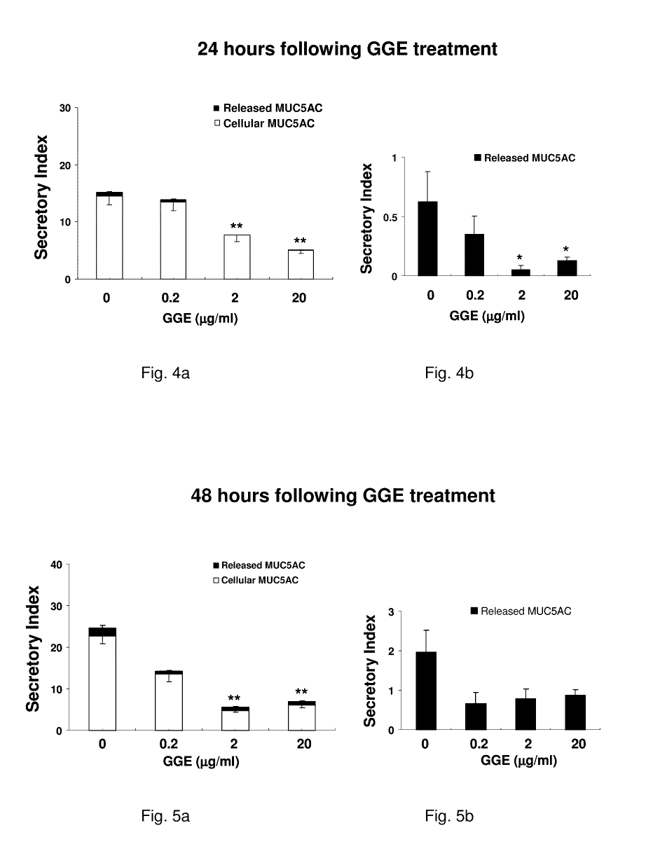

Twenty-four hour treatment with either 2 μg/mL or 20 μg/mL of guaifenesin significantly suppressed mucin release (FIG. 4b) as well as mucin production (FIG. 4a).

Treatment with guaifenesin (2 μg/mL and 20 μg/mL) for 48 hours significantly (p<0.01) suppressed the production of mucins (FIG. 5a). However, the amount of mucin released during this period did not seem to be significantly affected.

Effect of Guaifenesin on Mucociliary Clearance:

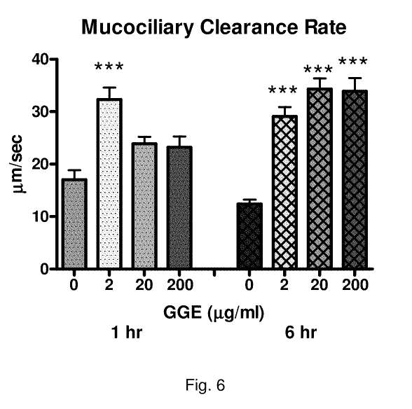

As shown in FIG. 6, guaifenesin appeared to increase the mobility of the cellular debris on the surface of cultures treated for 1 hr, but there was little evidence of a dose-response and in fact, only the effect of 2 μg/ml was statistically significant. However, at the 6 hr time point, there was a strong trend to a dose response and movement of the surface material for all three concentrations tested was significantly faster than the control as illustrated in FIG. 6.

EpiAirway cultures were treated with the indicated concentrations of guaifenesin for 1 or 6 hrs. Mucociliary clearance was assessed by the rate of movement of endogenous debris on the surfaces. *** indicates significantly different from the control cultures at the same time, p<0.005.

Viability:

There was no adverse effect on the viability of the cells as indicated by the WST assay. In fact, there appeared to be a trend to increased metabolic activity in the cells treated with guaifenesin, however this did not reach statistical significance. Data from one of the three replicate experiments is shown below.

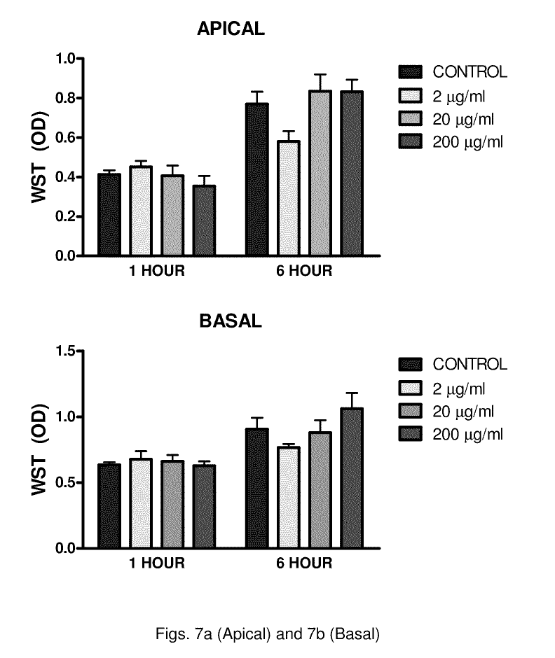

As shown in FIGS. 7a and 7b, EpiAirway cultures were treated with the indicated concentrations of guaifenesin for 1 or 6 hr. Metabolic activity was assessed using the WST assay, separately added to the apical or basal surfaces of the cultures.

Rheology:

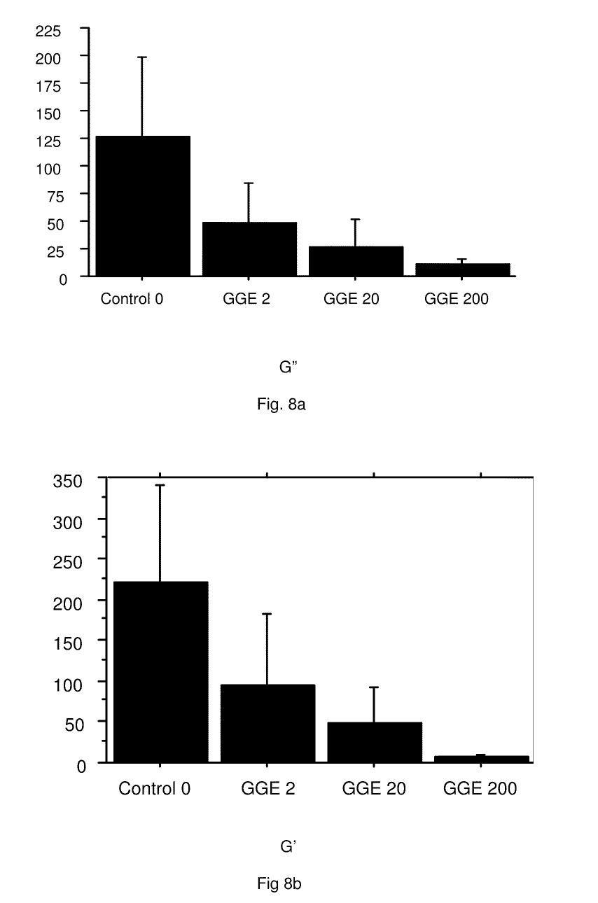

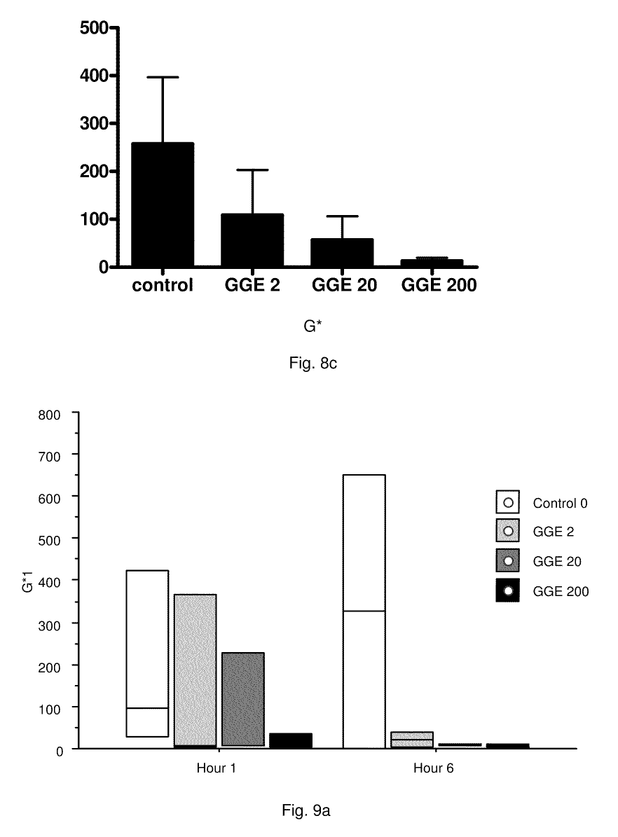

A total of 96 specimens from 5 sets of experiments were analyzed. The mucus from the first four experiments was received at ambient temperature and analysis of rheology of these samples showed extreme heterogeneity and the rheologic sweep curves obtained were consistent with degradation. The results shown in FIGS. 7 and 8 are therefore derived from the 22 specimens received from batch five. All specimens were non-Newtonian, viscoelastic gels.

The results demonstrate a significant guaifenesin dose-dependent decrease in viscosity, elasticity, and complex modulus (G*) of specimens at 1 hour (p<0.05) and especially at 6 hours (p<0.01) when measured at 1 rad/s or roughly ciliary frequency but not significantly at 100 rad/s corresponding to cough.

Mucus Rheology. FIG. 8a: G″ viscosity, FIG. 8b: G′ elasticity, FIG. 8c G* mechanical impedence (vector sum of viscosity and elasticity). Data shown are the mean and standard error of data from the 1 and 6 hr time points combined.

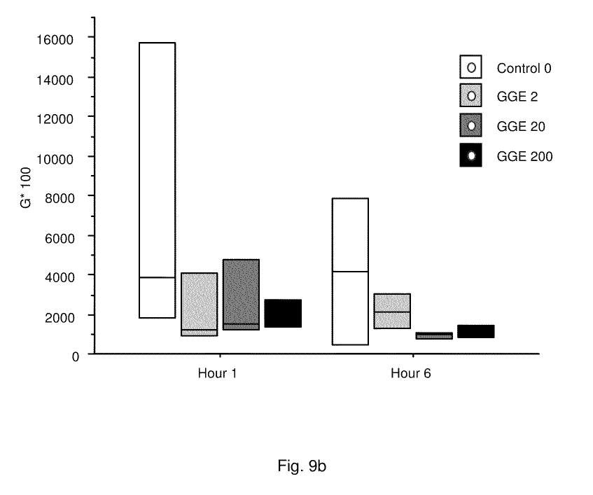

G*: vector sum of viscosity and elasticity, at 1 rad/s (FIG. 9a) and 100 rad/sec (FIG. 9b), segregated by time as well as dose.

In all three treatment time periods (6, 24 and 48 hours), guaifenesin at both 2 μg/mL and 20 μg/mL suppressed the production of mucins by NHBE cells grown on an air/liquid interface. Likewise, treatment with both 2 μg/mL and 20 μg/mL of guaifenesin for 24 hours showed a significant (p<0.05) decrease in mucin release.

To address the effects of guaifenesin on mucociliary clearance, mucociliary transport rates were measured. The purpose of these experiments was to investigate potential alterations in mucociliary clearance induced by exposure of differentiated primary human tracheo-bronchial epithelial cells to Guaifenesin. The original plan was to deposit aerosolized 1 μm diameter fluorescent microspheres on the surface of the cultures using a nebulizer. However, for reasons that are unclear, although the microspheres could be identified on the cultures, there was movement in only a very few of the cultures, despite clear movement of the endogenous cellular debris. A switch to collecting video of the endogenous debris was made.

Viscosity (loss modulus) is the loss of energy from a rheologic probe or applied stress and thus the resistance to flow. Elasticity (storage modulus) is the recoil energy transmitted back to the probe. The complex modulus, G*, is also known as the mechanical impedance. As the vectoral sum of the storage and loss moduli, G* measurement indicates resistance to deformation. Viscoelasticity is a property of non-Newtonian fluids (gels). Dynamic viscoelasticity measures the strain response of mucus to an applied stress. Because mucus is subjected to both low stress (ciliary beat) and high stress (cough) conditions, we measure the strain developed in response to a dynamic stress.

These results are consistent with the secretions taken from the differentiated cells being mucus gels. Although degradation of specimens from experiments 1-4 produced inconsistent results suggesting degradation (raw results all available on request), those from the final set of experiments were well preserved and the results were robust. The decrease in complex modulus paralleling that of the viscosity (loss modulus) would be consistent with the increased ciliary transport. The rheologic characteristics of these specimens suggested a goblet cell origin with viscosity approximately equal to elasticity, rather than a submucosal gland secretion where the elasticity is generally greater than viscosity. These results are consistent with the reported structure of the EpiAirway cultures. It will be informative to compare these results with those from human tissue explants exposed to guaifenesin.

Guaifenesin suppressed mucin production from confluent human bronchial epithelial cells grown on an air-liquid interface in a dose-dependent manner in vitro at concentrations that are clinically relevant. The reduction in mucus production correlated with increased mucociliary transport and decreased viscoelasticity of the mucus.

Further modifications or improvements cane be made without departing from the scope of the invention herein described.

What is claimed is:

1. A method of inhibiting mucus secretion in an individual comprising administering an effective amount of a composition comprising guaifenesin.

2. A method as claimed in claim 1, wherein the composition comprises from approximately 600 mg-1200 mg of guaifenesin.

3. A method as claimed in claim 2, wherein the composition comprises approximately 600 mg of guaifenesin.

4. A method as claimed in claim 2, wherein the composition comprises approximately 1200 mg of guaifenesin.

5. A method as claimed in claim 1, wherein administering an effective amount of a composition comprising guaifenesin comprises administering the composition as a tablet.

6. A method as claimed in claim 1, wherein administering an effective amount of a composition comprising guaifenesin comprises administering the composition as a powder.

7. A method as claimed in claim 1, wherein administering an effective amount of a composition comprising guaifenesin comprises administering the composition as a capsule.

8. A method as claimed in claim 1, wherein administering an effective amount of a composition comprising guaifenesin comprises administering the composition as a liquid.

9. A method as claimed in claim 1, wherein administering an effective amount of a composition comprising guaifenesin comprises administering the composition as a liquigel.

10. A method as claimed in claim 1, wherein the mucus secretion is produced in the upper respiratory tract of an individual.

11. A method as claimed in claim 1, wherein the composition further comprises one or more active agents.

12. A method as claimed in claim 11, wherein the one or more active agents are selected from the group consisting of an antitussive, a decongestant, and an antihistamine.

13. A method as claimed in claim 12, wherein the antitussive comprises dextromethorphan hydrobromide.

14. A method as claimed in claim 12, wherein the decongestant is selected from the group consisting of phenylephrine hydrochloride, pseudoephedrine hydrochloride and ephedrine.

15. A method as claimed in claim 12, wherein the antihistamine is selected from the group consisting of chlorpheniramine maleate, brompheniramine maleate, phenindamine tartrate, pyrilamine maleate, doxylamine succinate, phenyltoloxamine citrate, diphenhydramine hydrochloride, promethazine, clemastine fumerate, and fexofenadine.

16. A method as claimed in claim 1, wherein the composition comprises an immediate release portion and a sustained release portion, such that the inhibition of mucus secretion is therapeutically achieved for a period of approximately 12 hours.

17. A method of treating an individual having a disease or condition characterized by increased mucin secretion with an effective amount of a composition comprising guaifenesin.

18. A method as claimed in claim 17, wherein the composition comprises from approximately 600 mg-1200 mg of guaifenesin.

19. A method as claimed in claim 18, wherein the composition comprises approximately 600 mg of guaifenesin.

20. A method as claimed in claim 18, wherein the composition comprises approximately 1200 mg of guaifenesin.

21. A method as claimed in claim 17, wherein administering an effective amount of a composition comprising guaifenesin comprises administering the composition as a tablet.

22. A method as claimed in claim 17, wherein administering an effective amount of a composition comprising guaifenesin comprises administering the composition as a powder.

23. A method as claimed in claim 17, wherein administering an effective amount of a composition comprising guaifenesin comprises administering the composition as a capsule.

24. A method as claimed in claim 17, wherein administering an effective amount of a composition comprising guaifenesin comprises administering the composition as a liquid.

25. A method as claimed in claim 17, wherein administering an effective amount of a composition comprising guaifenesin comprises administering the composition as a liquigel.

26. A method as claimed in claim 17, wherein the mucus secretion is produced in the upper respiratory tract of an individual.

27. A method as claimed in claim 17, wherein the composition further comprises one or more active agents.

28. A method as claimed in claim 27, wherein the one or more active agents are selected from the group consisting of an antitussive, a decongestant, and an antihistamine.

29. A method as claimed in claim 28, wherein the antitussive comprises dextromethorphan hydrobromide.

30. A method as claimed in claim 28, wherein the decongestant is selected from the group consisting of phenylephrine hydrochloride, pseudoephedrine hydrochloride and ephedrine.

31. A method as claimed in claim 28, wherein the antihistamine is selected from the group consisting of chlorpheniramine maleate, brompheniramine maleate, phenindamine tartrate, pyrilamine maleate, doxylamine succinate, phenyltoloxamine citrate, diphenhydramine hydrochloride, promethazine, clemastine fumerate, and fexofenadine.

32. A method as claimed in claim 17, wherein the composition comprises an immediate release portion and a sustained release portion, such that the inhibition of mucus secretion is therapeutically achieved for a period of approximately 12 hours.
